(19) United States
(12) Patent Application Publication
(10) Pub. No.: US 2006/0233709 A1
(43) Pub. Date: Oct. 19, 2006

(54) Method for identifying novel treatments of inflammatory disease in the gut

(76) Inventors: Paul Goldsmith, Cambridge (GB); Angeleen Louise Fleming, Cambridge (GB)

Correspondence Address: KLARQUIST SPARKMAN, LLP, 121 SW SALMON STREET, SUITE 1600, PORTLAND, OR, 97204, US

(21) Appl. No.: 10/543,787

(22) PCT Filed: Jan. 28, 2004
(86) PCT No.: PCT/GB04/00349

§ 371 Date: Jan. 30, 2006

(30) Foreign Application Priority Data
Jan. 28, 2003 (GB) 0301972.6

(51) Int. Cl. A61K 49/00; A01K 67/027

(52) U.S. Cl. 424/9.2; 800/20

(57) ABSTRACT

Model fish and use thereof for screening for the presence of gut inflammatory disease, especially in zebrafish. Induction of the disease state and visualization of the gastrointestinal tract in living zebrafish. Visualization of the inflammatory state in vivo facilitates screening for compounds that can be used in treatment of inflammatory disease of the gut or genetic mutations that rescue or suppress the disease phenotype.

The present invention relates to a novel method of analyzing gut function in a living animal and subsequently utilisng this for the assessment and screening of a disease state. In particular, the present invention relates to a novel method for screening for the presence of inflammatory disease in a living animal. This is achieved through the induction of an inflammatory state in an observable tissue, in particular the gut of a fish, in particular a zebrafish. A method for both the induction of the disease state and the visualization of the gastrointestinal tract in living zebrafish is described. Visualization of the inflammatory state in vivo facilitates screening for compounds that can be used in the treatment of inflammatory bowel disease or genetic mutations that ‘rescue’ or suppress the disease phenotype.

Inflammation is a major component of numerous diseases. One common example of an inflammatory disease is Inflammatory Bowel Disease (IBD). The two major forms of IBD, Crohn's disease and ulcerative colitis, are common causes of gastrointestinal morbidity in Western Europe and North America. Genetic and environmental factors are important in both disease susceptibility and as determinants of disease progression.

Models of IBD have been created in mammals through the administration of pro-inflammatory agents to the gut. Problems with this approach include difficulties with administration and accuracy of dosing. Also, the agent typically only reaches one part of the gut. Furthermore, there is no easy way to assay the presence or severity of disease in the living animal, or to assay for modulators of the inflammatory state in a high-throughput fashion. The present invention describes novel and inventive solutions to these problems.

Administration of a pro-inflammatory agent, in particular picrylsulfonic acid (PSA), otherwise known as trinitrobenzene sulphonic acid (TNBS), to embryo medium at an appropriate dose and for an appropriate duration, in which are contained embryonic zebrafish an appropriate age results in inflammatory bowel disease, as determined by detailed histological analysis, including electron microscopic analysis. The fact that the agent is added to the water ensures equal exposure of all embryos to the same dose without repeated administration. Moreover, the disease state is induced throughout the entire gut.

Another pro-inflammatory agent that may be used is dextran sulphate sodium (DSS).

In order to screen for genetic suppressers of a disease it is desirable to be able to induce the disease state in a temporally controlled fashion to wild type embryos. This invention allows this. It is also important to be able to screen rapidly for the disease phenotype. This invention achieves this through the administration of a fluorescent dye to the embryo medium. The dye is swallowed by the fish and fills the entire gut. When viewed under a fluorescent microscope, the crypts and villi of the gut are seen.

Additionally, as the live fish is being visualised, waves of peristalsis can be observed. In contrast, in the disease state, the peristalsis is lost, as in human inflammatory conditions. The gut is dilated, with loss of the crypts and villi. The visualisation is very rapid, not requiring any histological processing, enabling a high-throughput screen to be carried out.

The invention allows for live and repeated visualisation of gut function. This permits study of both normal gut function and motility, as well as a variety of disease states and physiological phenomena, including inflammatory bowel disease, irritable bowel syndrome, nausea and vomiting, gut kinesis, constipation and diarrhoea, chemotherapy induced colitis and bowel stem cell function.

The invention also allows for the assessment of nausea, vomiting and gut motility.

This system is also amenable to high-throughput screening of therapeutic compounds. As an entire animal is being screened, optimal combinations of several possible anti-inflammatory agents may be screened together.

The following is a description of one preferred method for the induction of disease and subsequent visualisation:

Induction of IBD with PSA

Stock Solutions

Picryl sulfonic acid (Sigma P2297) stored as a powder at 4° C. Stock solution of 1 mg/ml PSA in embryo medium. Stored at 4° C. for maximum of 2 months.

Screening Protocol

1) Zebrafish larvae were immersed in embryo medium containing 75 μg/ml picryl sulfonic acid from 3 d.p.f. Abnormal gut architecture is observed from 5 d.p.f.

2) For in vivo examination of gut architecture:

a) Add a forceps pinch of quercetin (Sigma Q0125) to a large Petri dish, or if using 96 well plate format, add 2 μl to each well (N.B. Quercetin is not water soluble).

b) Quercetin labelling can be performed from 5 d.p.f. onwards. Once quercetin has been added, it is necessary to change medium daily.

c) For large scale screening (e.g. 96 well plate format), quercetin is added early on 8 d.p.f, larvae scored late 8 d.p.f, or at early 9 d.p.f.

d) For in vivo observations: Anaesthetise larvae by addition of MS222. When screening large samples (e.g. 96 well plate), anaesthetise one well at a time. View quercetin labelling using the fluorescence microscope. Staining can be seen using the FITC and GFP filter sets.

The relevance of this model is demonstrated by the rescue of the disease phenotype with 2 agents known to rescue human IBD
prednisolone and salicylic acid.

Rescue of IBD with prednisolone

Protocol:

1) IBD was induced with PSA as described above.

2) Prednisolone stock (Sigma M0639) was made up as 50 μg/ml stock in embryo medium.

The stock was stored at 4 ° C. for a maximum of 2 months.

3) IBD was induced by exposure of embryos to PSA from 3 d.p.f. to 5 d.p.f. The medium was changed at 5 d.p.f.

Rescue of the IBD phenotype was seen.

Rescue of IBD with 5-ASA

Protocol:

1) IBD induced with PSA as described above.

2) 5-ASA stock (Sigma A3021) was made up as 2 mg/ml stock in embryo medium. pH was adjusted to neutral allow the 5-ASA to go into solution. The stock was stored in the dark at 4° C. for a maximum of 2 months.

3) IBD was induced by exposure of embryos to PSA from 3 d.p.f. to 5 d.p.f. The medium as changed at 5 d.p.f. and replaced with PSA co-administered with prednisolone +5-ASA . The assay was kept in the dark.

Rescue of the IBD phenotype was seen.

The characteristic immunological reaction to the inflammatory state, determined in the detailed histological analysis, allows this model to be used as a rapid, in vivo model of inflammation in other human diseases as well as IBD.

Mucosecretory disease is poorly represented in animal models. The transdifferentiation of goblet cells observed in the posterior intestine of zebrafish may be used to provide a model for the study of mucosecretory disease. This model can be used to elucidate the factors that drive goblet cell transdifferentiation and also to screen for factors that suppress their differentiation. It is thus also of relevance to the study of cancerous states, and in particular metaplasia.

Irritable bowel syndrome (IBS) is an umbrella term for the presence of a collection of abdominal symptoms for which an alternative pathological cause cannot be found. Common symptoms include abdominal pain, bloating, and altered bowel habit. Most cases are probably due to disordered motility. The complex of nerves in the bowel wall control motility, so IBS can be considered a disorder of the enteric nervous system. It is the commonest condition seen by gastroenterologists, affecting up to 25% of the population occasionally, a further 25% of whom will have symptoms severe enough to prompt medical referral. In approximately 50% an improvement in symptoms over 12 months is seen. In others, chronic intermittent symptoms are more typical. No treatment is universally successful. Approximately 30% of patients respond to any particular drug, although the efficacy of this drug may vary with time. A constipating agent, such as loperamide, amitriptyline or codeine, is used if diarrhoea is prominent, and a high-fibre diet if constipation is a symptom. Anticholinergics such as mebeverine have a useful antispasmodic action. They are very effective for a few patients, partially effective for many, but ineffective for others. There is a need for both more effective therapies, in particular to normalise gut motility, and for models of the disease state to help identify and predict the efficacy of candidate therapies. We have developed strategies to visualise gut motility in vivo in a high-throughput fashion. Screening can be focused to measure a variety of parameters including transit time, strength and frequency of contractions, co-ordination of contraction. Such screens will enable us identify agents which reverse dysmotility and that can be developed for the treatment of IBS.

We have observed that certain stimuli, for example rapid pipetting of the fish water, lead to reverse peristalsis and regurgitation, a finding we have also observed when fish are fed a food pellet unsuitable for their age. This therefore makles this model suitable for analyzing nausea and vomiting, antiemetics and pro-kinetics, looking for agents which would not be good drug candidates as they would lead to vomiting, or looking for agents which suppress the response to an emetic and would therefore be a candidate anti-emetic, or agents which accelerate peristaltic waves and would thus be good pro-kinetic agents.

Inflammation is a major component of numerous diseases all of which are characterised by immunological reaction to the inflammatory state. The disease changes observed in IBD in zebrafish can be extrapolated to other inflammatory conditions, allowing this model to be used as a rapid, in vivo model of inflammation in other human diseases as well as IBD.

As noted, the invention allows to visualize gut function in a live animal in a high throughput fashion amenable to screening, for example by seeing peristaltic waves, crypts and villi.

The invention is useful for looking at conditions, including, for example, gut motility per se, irritable bowel syndrome, the effect of anti-emetics, constipation, and other gut disorders, e.g. celiac disease.

The invention relates to a method of inducing inflammatory bowel disease in the model, with visualizing the output and screening in live fish, e.g. by means of altered peristalsis, abnormal morphology (dilation, loss of normal crypt and villi structure), and in fixed specimens, e.g. by determining TNFα levels, looking at H&E sections, by mast cell counting and by determining goblet cell numbers and their presence in which regions of the gut.

Provided by the present invention are fish disease models which are not only representative of the underlying disease, but are also particularly amenable for use in subsequent screening. This allows in turn the identification of a human or other therapeutic.

The invention also provides a means for assaying faecal throughput and therefore a measure of food consumption and absorption, and consequently a useful assay for satiety, food preference, absorption and obesity.

Additional methods for the induction of intestinal damage and Inflammation are provided within aspects and embodiments of the present invention.

We have observed that addition of sudan black in ethylene glycol to the embryo medium causes shedding from the gut epithelium and bleeding as a result of disrupted intestinal barrier function. These disease changes cause the lumen of the gut to appear red. These changes can be observed in the living animal using a simple low resolution dissecting microscope and hence are amenable to high throughput screening.

Observations on the induction of this disease state are based on and may employ in vivo, histological and EM analysis.

The invention is generally applicable to any of a variety of diseases and disorders, and a range of examples is specifically set out herein.

At all stages the agent altering the phenotype may be by means of application of a drug, protein, antibody or genetic alteration, or other manipulation.

The present invention provides means, specifically a fish model as claimed and disclosed herein, and methods as claimed and disclosed.

The zebrafish is an organism which combines many of the advantages of mammalian and invertebrate model systems. It is a vertebrate and thus more relevant in models of human disease than Drosophila or other invertebrates, but unlike other vertebrate models it can be used to perform genetic screens.

A number of peer reviewed papers highlight and validate the use of zebrafish as a species in which to model human disorders. [Dooley K and Zon LI (2000) Current Opinion in Genetics and Development 10 : 252-6; Barut BA and Zon LI (2000) Physiological Genomics 13: 49-51; Fishman MC (2001) Science 294: 1290-1].

The use of vertebrates offers the opportunity to perform sophisticated analyses to identify genes and processes involved in disease.

The inventors have appreciated that zebrafish offer the unique combination of invertebrate scalability and vertebrate modelling capabilities. They develop rapidly, with the basic body plan already having been laid out within 24 hours of fertilization. Moreover, their ex-utero development within a transparent capsule allows the easy in vivo visualisation of internal organs through a dissecting microscope. Many disease states can be modelled within the first week of life, at which time the embryos are only a few millimetres long and capable of living in 100 μl of fluid. This permits analysis of individual embryos in multi-channel format, such as 96 well plate format. This is particularly useful for drug screening, with many chemicals being arranged in 96 well plate format.

Alternatively, a population of fish in a petri dish or a tank may be employed. A population of fish may be treated together, and may be tested together, e.g. via addition of one or more or a combination of test substances to the water.

The zebrafish has a short maturation period of two to three months and is highly fecund, with a single pair of adults capable of producing 100 to 200 offspring per week. Both embryos and adults are small, embryos being a few mm and adults 2-3 cm long. They are cheap and easy to maintain. The ability to generate large numbers of offspring in a small place offers the potential of large scalability.

In addition to Zebrafish, other fish such as fugu, goldfish, medaka and giant rerio are amenable to manipulation, mutation and study, and use in aspects and embodiments of the present invention as disclosed herein.

In a further aspect, the present invention provides a method of making a fish model as disclosed, useful in or for use in a screen as disclosed herein and discussed further below.

In mutation a fish to determine the effect of such mutation on disease phenotype, a number of approaches may be taken.

Such a method may comprise providing a gene construct wherein a coding sequence of a disease gene is operably linked to a promoter that has the desired inducibility and/or tissue specificity, in the fish, introducing the gene construct into a fish embryo, causing or allowing the gene construct to integrate into the fish embryo genome, and growing the fish embryo into a viable fish.

A viable and reproductive fish may mate with one or more other fish, establishing a line of fish, e.g. zebrafish, transgenic for the gene construct comprising the disease gene operably linked to, and under regulatory control of, the promoter. A line of such fish, e.g. zebrafish, is useful in screens as disclosed.

In order to introduce a gene into a fish embryo, e.g. a zebrafish embryo, a gene construct is made, using techniques available to those skilled in the art. The construct may be released from a vector by restriction digest, and gel purified, for example by elution in 1×TE (pH8.0) and dilution to a working concentration of 50-100 μg/ml KCl containing a marker dye such as tetramethyl-rhodamine dextran (0.125%). Typically, 1 to 3 nl of this solution may be injected into single celled zebrafish embryos. Several thousand embryos may be injected.

Injected embryos are grown up and then mated with each other or to a non-transgenic wild-type fish. Transmission of the transgene to the subsequent generation is usually mosaic, ranging from 2 to 90%. At least 100 offspring are typically analysed to establish whether the founder fish carriers the transgene.

Fish demonstrating a desired phenotype and/or genotype may be grown up and may be mated with wild-type fish. The parents and offspring may be matched and the offspring similarly assessed for phenotype and/or genotype. Those offspring with a particular phenotype, and hence likely germline transmission of an integrated disease gene construct, can be selectively bred. Some of the offspring may be sacrificed for more detailed analysis, e.g. to confirm the nature of the autoimmune disease. This analysis may include in situ hybridisation studies using sense and anti-sense probes to the introduced gene to check for expression of the construct in cells of the fish, anatomical assessment such as with plastic sections to check for an effect on tissue or cells, and terminal deoxyuridine nucleotide end labelling (TUNEL) to check for apoptotic cell death in cells.

Families from which fish with the appropriate characteristics came may be maintained through subsequent generations. This maintenance then allows this new mutant strain to be entered into a secondary screen in accordance with further aspects of the invention.

A gene such as a disease gene sequence (e.g. heterologous to the fish e.g. zebrafish) to be employed in aspects and embodiments of the present invention may employ a wild-type gene or a mutant, variant or derivative sequence may be employed. The sequence may differ from wild-type by a change which is one or more of addition, insertion, deletion and substitution of one or more nucleotides of the sequence shown. Changes to a nucleotide sequence may result in an amino acid change at the protein level, or not, as determined by the genetic code.

Some aspects of the invention involve genetic rescue of an induced phenotype. Fish such as Zebrafish are particularly amenable to genetic rescue experiments.

Mutagens such as ethylnitrosourea (ENU) may be used to generate mutated lines for rescue screening, in either the F1-3 (for dominant) or F3 (for recessive) generations. (It is only by the third generation that recessive mutations can be bred to homozygosity.) ENU introduces point mutations with high efficiency. Retroviral vectors may be used for mutagenesis, and although they are an order of magnitude less effective than ENU they offer the advantage of rapid cloning of a mutated gene (see e.g. Golling et al.(2002) Nat Genet 31, 135-40). Mariner/Tc family transposable elements have been successfully mobilised in the zebrafish genome and may be used as mutagenic agents (Raz et al. (1998) Curr Biol 8, 82-8). ENU remains the most efficient and easy method available at the moment, and so is preferred for now.

Rescue strains are then created and the underlying genes mapped.

The mapping of mutant genes is comparatively easy. The density of markers on the fish genetic map, for example, is already considerably greater than that of the mouse map, despite the relatively recent popularity of zebrafish. Consult the harvard website on zebrafish, findable using any available web browser using terms “zebrafish” AND “harvard”, currently (28 Nov. 2002 and 22 Jan. 2004) found at (http://zebrafish.mgh.harvard.edu/mapping/ssr_map_index.html. The Sanger Centre has is sequencing the zebrafish genome with sequence currently (28 Nov. 2002 and 22 Jan. 2004) published at www.ensembl.org/Danio_rerio/. The site can be found using any web browser using terms “danio rerio” and “Sanger” or “ENSEMBL”. Around 70,000 ESTs have been identified and are being mapped on a radiation-hybrid map.

Another strategy for introducing effects, which may be random, on an aspect of behaviour or physiology in accordance with the present invention, is to down-regulate the function or activity of a gene, for instance employing a gene silencing or antisense technique, such as RNA interference or morpholinos. These can be either targeted against candidate genes, or generated against-an array of genes as part of a systematic screen. It is relatively easy to inject RNA, DNA, chemicals, morpholinos or fluorescent markers into fish embryos, including zebrafish embryos, given their ex utero development.

A morpholino is a modified oligonucleotide containing A, C, G or T linked to a morpholine ring which protects against degradation and enhances stability. Antisense morpholinos bind to and inactivate RNAs and seem to work particularly well in zebrafish. Some disadvantages with this approach include the a priori need to know the gene sequence, the need to inject the chemical into the early embryo, potential toxic side effects and the relatively short duration of action. Additionally, they knock down the function of a gene, and thus do not offer the same repertoire of allele alterations as point mutations.

A further strategy for altering the function of a gene or protein as part of an in vivo screen, coupled to any of the various other components of the screening strategy disclosed herein, is to generate transgenic lines expressing protein aptamers, crossing these with the disease lines, or inducing disease by other means, then assaying for an altered disease state. Protein aptamers provide another route for drug discovery [Colas, 1996] but the ability to assay their effectiveness in vivo in accordance with the present invention markedly increasing their usefulness beyond in vitro screening methods.

Of course, the person skilled in the art will design any appropriate control experiments with which to compare results obtained in test assays.

In various further aspects, the present invention thus provides a pharmaceutical composition, medicament, drug or other composition comprising a suppressor gene or other gene or gene product or substance found to affect the disease gene of interest or suppression of the disease gene of interest, the use of such a material in a method of medical treatment, a method comprising administration of such a material to a patient, e.g. for treatment(which may include preventative treatment) of a medical condition, use of such a material in the manufacture of a composition, medicament or drug for administration for such a purpose, e.g. for treatment of a proliferative disorder, and a method of making a pharmaceutical composition comprising admixing such a material with a pharmaceutically acceptable excipient, vehicle or carrier, and optionally other ingredients.

One or more small molecules may be preferred therapeutics identified or obtained by means of the present invention. However, the invention may be used to identify appropriate targets for antibody mediated therapy, therapy mediated through gene targeting or protein targeting, or any of a variety of gene silencing techniques, including RNAi, antisense and morpholinos.

Alternatively, instead of or as well as an attempt to rescue the phenotype through the induction of genetic mutations, rescue may be achieved through application of a test substance, e.g. one or more chemicals. In this situation, all of the above methods need not require the mutation step for rescue (but will still require the mutation step if this is part of the procedure for induction of the disease state).

In a further aspect of the present invention, a fish in which one or more symptoms of a condition has been induced, may be treated with a test substance to screen for a substance capable of affecting the development of the condition. The effect of the test substance may be assessed by comparing an aspect of behaviour or physiology of treated fish with that aspect of behaviour or physiology of untreated fish to identify any treated fish with altered behaviour or physiology compared with an untreated fish, thereby to identify a test substance that affects development of the autoimmune disease state.

The present invention provides means, specifically model fish for use in methods of screening for a test substance which when administered ameliorates symptoms of inflammation or the autoimmune component of IBD.

Fish may be treated with a test substance in a number of ways. For example, fish may be contacted with the test substance, it may be touched or rubbed on their surface or injected into them.

A further advantage of fish, especially zebrafish is the fact they live in water. This makes administration of test substances easy as they may be added to water in which the fish are. Zebrafish and other fish also readily absorb chemicals. The effective concentration of chemicals in the water often equates to the effective plasma concentration in mammals.

Different test substances-may be added to each well of a multi-well plate, such as a 96 well plate, to identify that test substance exhibiting a beneficial or deleterious effect. There may be one or multiple fish in each well exposed to the test substance.

Moreover, the inventors have discovered that zebrafish are also DMSO (dimethyl sulphoxide) tolerant. This is important as DMSO is used as a solvent to dissolve many drugs. The inventors have established that zebrafish can tolerate 1% DMSO. Thus, a candidate drug or other test substance may be dissolved in DMSO and administered to zebrafish by adding to the fish water to give a final concentration of DMSO of at least up to 1%. This is employed in various preferred aspects and embodiments of the present invention.

The test substance may be added prior to the onset of the disease phenotype or concurrent with the onset of the disease phenotype. Preferably the test substance may be added subsequent to the onset of the disease phenotype.

The same test substance may be added to different wells at a different concentration. For example, test substance 1 may be added to well A1 at a concentration of 1 mM, to well A2 at a concentration of 100 uM, to well A3 at a concentration of 10 uM, to well A4 at a concentration of 1 uM and to well A5 at a concentration of 0.1 uM. Then test substance 2 to well B1 etc. The panel of test substances may be known drugs or new chemical entities.

Additionally, the test substances may be added in combination. For example, well A2 may contain test substance 1 and 2, well A3 test substance 1 and 3, well B2 test substance 2 and 3. Alternatively, every well may contain test substance x, with individual wells containing a panel of additional test substances.

In other options, a population of fish in a petri dish or a tank may be employed and treated together, e.g. via addition of one or more or a combination of test substances in the water.

Thus, zebrafish enable the entire biological pathway of a vertebrate to be screened in a high-throughput fashion.

The present invention in certain aspects and embodiments provides for screening for and preferably identifying or obtaining a substance that provides a synergistic combination with another substance, or for screening for and preferably identifying or obtaining two or more substances that together provide an additive or synergistic combination. Clinical benefit is often derived from synergistic combinations of drugs. Use of an in vivo system in accordance with the present invention allows for identification of such synergistic combinations.

Thus, in certain embodiments the invention comprises treating the fish, as discussed, with two or more substances, at least one of which is a test substance, and comparing the effect of the two or more substances in combination to determine the optimum effect (whether simultaneously or sequentially applied) on an aspect of behaviour or physiology with the effect of either or both of the two or more substances when. applied individually or alone. Either all (or both) of the substances applied may each be a test substance, or one of the substances may be a drug known to have a beneficial effect in the disease that is the subject of the model, or at least an effect in the treated fish model.

The invention thus provides for screening for and preferably identifying or obtaining a substance that provides an additive effect to a known drug or a synergistic effect with the known drug. It also provides for screening for and preferably identifying or obtaining a combination of two or more substances that provide a synergistic effect, compared with the effect of the two substances when employed individually or alone.

Add-on therapies are useful because it is difficult to conduct clinical trials in which an existing drug is withdrawn from a patient and replaced with a new drug. The patient is deprived of a drug which has at least got some proven efficacy and some confidence in its side-effect profile. Additionally, the patient will be vulnerable to their disease during the phases of withdrawal of the existing drug and build up of the test drug. For example, relating to the example given earlier, many patients with IBD will be already taking 5-aminosalicylic acid and/or prednisolone, so it is very useful to be able to tell if a genetic mutation or test compound is additive or synergistic to the current therapy. This invention facilitates this.

In addition to a test substance, the fish may be a mutated animal rather than a wild-type animal. It is then possible to assay for interacting effects, either beneficial synergistic effects, or deleterious effects, of the mutation plus the test substances. Alternatively, the analysis may be of the known therapeutic agent and the genetic mutation to discover either a new drug target of benefit in combination with the known drug, or a genetic marker of use in predicting which patients are most likely to benefit (or not benefit) from prescription of the known drug.

In another embodiment, a combination of potential immunosuppressive agents is administered to a fish having one or more symptoms of an inflammatory disease, which may be generated as disclosed herein, to assess whether the combination is more effective than either of the individual agents.

For example, there are a variety of immunosuppressive agents, either in clinical trials or currently prescribed. For the sake of this example, assume there are 11 drugs to be tested. It is possible that the various drugs act at different pinch points in biological pathways and that by judicious co-prescribing, an optimal combination may be found that is better than any drug alone, whilst with no worse a side effect profile. It would be very difficult to do clinical trials, or indeed mammalian studies to determine the optimum combination. The present invention allows this.

The present invention also provides for screening for and preferably identifying or obtaining a substance that ameliorates one or more side effects of an active substance, e.g. a therapeutically active substance. There are many drugs which have been discontinued in clinical trials, or are marketed but infrequently prescribed, not because they are not therapeutically effective, but because their side-effect profile is limiting. The side-effects may be relatively benign, but significant to the patient, such as renal damage (e.g. cyclosporin). It is desirable to allow the administration of such drugs, with proven beneficial effects, through the co-administration of an additional agent to improve the side-effect profile.

In accordance with the present invention, such agents are screened for in fish in which administration of the active substance induces a side-effect or other phenotype reflective or indicative of a side-effect. Thus in embodiments of the invention, an active agent is administered to fish having one or more symptoms of an inflammatory disease and the side-effect of other phenotype is assessed for such animals when subjected to one or more test substances. This does not require a priori knowledge of action of the co-administered agent. In other embodiments, agents that achieve the desired therapeutic effect with a reduction of side-effects can be screened for and preferably identified or obtained by means of assessment of disease phenotype and side-effect phenotype. As with other aspects and embodiments of the present invention, this may involve co-administration of a primary compound together with either a battery of candidate substances, or together with randomly induced genetic mutation. With the latter approach, i.e. mutation, subsequent steps are needed to identify the appropriate co-therapeutic following identification of fish with a mutation that provides an ameliorative effect.

A diverse library of drug-like compounds, such as the LOPAC library (Sigma) may be used, or the Chembridge PHARMACOphore. diverse combinatorial library. Other targeted libraries against particular targets classes may be used, such as ion channel libraries or G protein libraries.

Still further provided by the present invention is a method of identifying mutations, genotypes, allelic variations, haplotypes and genetic profiles associated with responsiveness to a therapeutic. There is an increasing move towards targeted prescribing, whereby the choice of therapeutic is influenced by genotyping the patient. Particular polymorphisms have been found to predict both the therapeutic effectiveness of a compound, and also the likelihood of suffering certain side effects. Such rationalised prescribing is cost-effective. It also makes clinical trials easier to run, as likely responders can be targeted, thus necessitating a smaller sample size to achieve statistical significance. However, for the moment, most drugs, both already prescribed or in development, do not have an appropriate test.

The present invention provides for assessing the effectiveness of various medications in combination with random genetic mutations to identify those mutations which either enhance or decrease the therapeutic effectiveness and/or alter the side effect profile. This allows for identification of genes, polymorphisms, mutations, alleles and haplotypes associated with a particular response to a drug or other treatment, enabling development of appropriate genetic assays in humans to permit rationalised prescribing.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility. Such means for screening for substances potentially useful in treating or preventing a disorder or disease is provided by fish in accordance with the present invention. Modifier genes, such as enhancer or suppressor genes identified using the invention, and substances that affect activity of such suppressor genes represent an advance in the fight against disease since they provide basis for design and investigation of therapeutics for in vivo use, as do test substances able to affect activity or effect of a treatment, and substances that affect activity or effect of expression of a disease gene in a fish.

In various further aspects the present invention relates to screening and assay methods and means, and substances identified thereby.

Whatever the material used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and. other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see US Patent No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer, also administration of naked DNA or RNA, by simple administration, e.g. injection, of nucleic acid such as a plasmid, for instance to muscle.

The application of a test substance may-then be as follows, in accordance with embodiments of the present invention:

1. A test substance is added to the fish either prior to the appearance of the disease state, at the time of induction of the disease state, or after the induction of the disease state. The first two situations are more likely to identify a prophylactic chemical, the latter a drug which reverts the disease state back to normal. The test substance may be a chemical and may be a random chemical administered in a high-throughput fashion to fish in 96 well plate format, or a selected chemical administered to a clutch of fish in a Petri dish.

2. The fish is then screened for deviation from the initial disease state.

The following additional steps are highly desirable in screening, and their use is provided by the present invention in preferred embodiments:

Rather than add a single chemical, a combination of chemicals is added. For instance, a known therapeutic agent may be administered to all fish at a dose at which a further beneficial effect could still be detected. A random chemical library is then added to fish and an incremental effect screened for.

A further embodiment allows for detection of augmentation of a particular drug through a particular mutation, as follows:

1. Induce genetic mutation through any of the above.

2. Induce disease state.

3. Administer test chemical.

4. Assess whether the combination of the mutation plus chemical is greater than either alone.

5. The mutated gene is then used as a beneficial target, as described above.

A further embodiment of the invention allows identification of genetic factors which help determine the appropriateness of a particular therapeutic agent for a given patient. If the mutation augments the effect of the drug, that mutation is searched for in human homologues. Patients with this mutation should be preferentially prescribed the drug. If the mutation leads to a deleterious effect or lack of effect, then patients should avoid this drug.

A further embodiment of the invention allows identification of genetic or chemical factors which help prevent the side effects of an otherwise toxic drug. The following is an illustrative embodiment, and may be applied in other contexts for other diseases:

1. Drug X has a beneficial effect on disease Y, but causes side effect Z.

2. An zebrafish model is created which responds to treatment with drug X, but with the added complication of side effect Z.

3. The treated fish are co-treated with a panel of chemicals, (or alternatively are mutagenised as a route to a drug target).

4. Those fish which no longer show the side effect, but still show the beneficial effects are selected. The chemical is then used as a co-agent in patients to allow the safer administration of drug X, (or alternatively the mutagenised gene is mapped and used to develop the co-agent).

A further embodiment of the present invention involves attempting to modify the initial phenotype through a protein aptamer, rather than through a genetic mutation of chemical means. For example, a method may be performed in accordance with the following:

1. A construct coding for the desired aptamer (or random constructs for random aptamers) is injected into embryos to generate lines expressing the aptamer.

2. These lines are then crossed to the disease-expressing lines, or alternatively the disease state is induced in these lines.

3. The lines are then tested for deviation from the expected or intial phenotype.

4. If deviation occurs, the aptamer has in vivo proof of action and is used to derive a therapeutic agent.

Having identified fish with a mutation that confers rescue on a disease phenotype, the following steps may be performed:

1. The human homologue of the zebrafish rescue gene is cloned.

2. The same type of mutation is introduced into the human homologue

3. The wild-type and mutated constructs are injected into the embryos.

4. The disease state is induced and assessed.

5. If the wild-type gene prevents the rescue, but the mutant gene retains it, this provides further evidence that the mutation is beneficial. However, a negative result does not necessarily rule out benefit.

6. The protein encoded by the human homologue is used for direct drug screens in vitro or directed in vivo screening.

Over the past decade, much of the progress in our understanding of Inflammatory Bowel Disease (IBD) has come through human genetic studies that have identified key susceptibility genes and loci (Parkes and Jewell, 2001; Watts and Satsangi, 2002). Such studies have demonstrated that gene-environment interactions determine both disease susceptibility and progression. Yet, while these findings have a clinical application in stratifying of patient groups for therapy, there has been little change in the treatment options for either Crohn's disease (CD) or ulcerative colitis (UC). Studies in animal models of colitis have also focused on genetic components of the disease. Mice lacking or over-expressing various genes in immune response and inflammatory pathways have demonstrated that de-regulation of many steps of the inflammatory response can lead to chronic inflammation in the intestine (Mueller, 2002). While these genetic models enable the identification of ‘pinch points’ in the pathogenesis of colitis, none are able to replicate all aspects of the pathological, clinical and histological changes seen in CD or UC in humans and hence are problematic in the identification of targets for future therapies. Here, we describe a method of analyzing gut function in a live vertebrate in a high-throughput, high content fashion applicable to emesis, pro-kinesis, IBS and IBD, amongst others. We also describe a method of inducing IBD in this model showing biological, pathological and clinical relevance to the human condition and the methods for and results of screening in this model

Inflammatory bowel disease (IBD) is a disease of multi-factoral but largely unknown aetiology characterized by chronic relapsing and remitting intestinal inflammation. The two major forms of IBD, Crohn's disease and Ulcerative Colitis, are common causes of gastrointestinal morbidity in Western Europe and North America (Calkins and Mendeloff, 1995). Genetic and environmental factors are important in both disease susceptibility and as determinants of disease progression. While the identification of susceptibility genes is of great value in the prediction of disease and defining patient populations for therapy, the encoded proteins are unlikely to useful targets for the design of novel therapies. As a result, the pharmaceutical industry relies largely on rodent models of IBD. Although there are many such models, none stand out as being truly representative of human disease. Traditionally, IBD is induced in rats and mice by the administration of pro-inflammatory agents to the gut. Problems with this approach include difficulties with administration and accuracy of dosing. In additions, the pro-inflammatory agent typically only reaches one part of the gut, and hence the disease is localized to only a small area of the intestine. The study of rodent models is further complicated by methods of analysis, as there is no easy way to assay the presence or severity of disease in the living animal, or to measure modulators of the inflammatory state in a high-throughput fashion. Here we describe a zebrafish model of IBD, in which region specific disease changes, relevant to the human disease, are seen throughout the length of the gut. Furthermore, since we are able to visualise such changes in the living animal, this model is amenable to high-throughput screening for the identification of novel therapies for IBD.

MATERIALS AND METHODS

Maintenance of Stocks and Collection of Embryos

Fish were reared under standard conditions (Westerfield, 1995). Embryos were collected from natural spawnings, staged according to established criteria (Kimmel et al., 1995) and reared in embryo medium (5 mM NaCl, 0.17 mM KCl,0.33 mMCaCl_{2,} 0.33 mM Mg₂SO_{4,} 10⁻⁵% Methylene Blue)

In Vivo Examination of Gut Architecture

In vivo labelling was achieved by rearing fish in a suspension of 0.05% quercetin (Sigma) for a minimum of 2 hours. To observe gut architecture and motility, larvae were anaesthetised larvae by immersion in 0.2 mg/ml 3-amino benzoic acid ethylester (MS222) (Sigma). Gut architecture and motility can be observed using the FITC and GFP filter sets.

Induction of IBD

A stock solution of 1 mg/ml Picryl sulfonic acid (Sigma) (trinitrobenzene sulfonic acid - TNBS) in embryo medium was used for the induction of IBD. The stock solution was stored at 4° C. for maximum of 2 months. IBD was induced by immersing zebrafish larvae from 3 days post-fertilisation (d.p.f.) in embryo medium containing 75 ug/ml picryl sulfonic acid (PSA) made fresh on the day of the assay.

Rescue of IBD with Prednisolone and 5-aminosalicylic acid

A 50 ug/ml stock solution of 6-α amethyl prednisolone (Sigma) in embryo medium was used in all prednisolone rescue experiments. A 2 mg/ml stock solution of 5-amino salicylic acid (5-ASA ) (Sigma) in embryo medium was used in all 5-ASA rescue experiments. The pH of the solution was adjusted to pH 7.5 using NaOH. The stock was stored in the dark at 4° C. for a maximum of 2 months. IBD was induced by exposure of larvae to 75 ug/ml PSA from 3 d.p.f. to 5 d.p.f. At 5 d.p.f., medium containing 75 ug/ml PSA was removed and replaced with 75 ug/ml PSA with in combination with prednisolone or 5-ASA . Prednisolone and 5-ASA were tested at a range of concentrations (1 to 25 ug/ml and 2 to 200 μg/ml respectively) to find the most effective rescuing dose. Larvae exposed to 5-ASA were kept in the dark as the solution is light sensitive. In vivo observations were performed as described at 8 d.p.f. after which larvae were fixed and processed for antibody staining or histology.

Screening for rescue of IBD with iNOS inhibitors, thalidomide and parthenolide

Stock solutions of a panel of iNOS inhibitors; 2,2′- Dithiobis(guanidinoethane, S-Methyl-L-thiocitrulline acetate, 1-(-2-Trifluoromethylphenyl)imidazole (Sigma) were made at 2 mg/ml in DMSO and stored at −20° C. A 50 mg/ml stock of thalidomide (Sigma) was made in DMSO. A50 mg/ml stock of parthenolide (Sigma) was made in DMSO. In control groups, IBD was induced by exposure of larvae to 75 μg/ml PSA from 3 d.p.f. to 8 d.p.f. Compounds with possible anti-inflammatory effects were tested by co-administration with PSA from 3 d.p.f. to 8 d.p.f. All compounds were tested at a range of concentrations (iNOS inhibitors from 20 ng/ml to 2 mg/ml; thalidomide and parthenolide from 50 μg/ml to 2 mg/ml and 10 ng/ml to 100 ng/ml respectively) to find the most effective rescuing dose from in vivo observations. In vivo observations were performed as described at 8 d.p.f. after which larvae were fixed and processed for antibody staining or histology.

Histology

Larvae were anaesthetised larvae by immersion in 0.2 mg/ml 3-amino benzoic acid (MS222) prior to fixation in Bouins Fixative (Fisher Scientific). Samples were fixed for 24 hours to 1 month at room temperature. Samples were dehydrated through a graded series of alcohols and Histoclear (National Diagnostics) then embedded in wax (Raymond A Lamb). 7 μm transverse or parasagittal (longitudinal) sections were cut using a Leitz microtome and mounted on Superfrost Plus slides (Scientific Lab Supplies) prior to staining with haematoxylin and eosin for basic histology or ethanol-based toluidine (0.25% toluidine blue in 70% ethanol) for the detection of mast cells (Gurr, 1962). Histology samples were viewed on a BX51 microscope (Olympus) and images were taken using a ColorView camera and AnalySis software (Olympus).

Mast Cell Quantification

Three sagittal sections were identified from each individual larva. Sagittal sections were identified as those in which the lumen of the gut was present at all levels from mouth to anus. Total number of mast cells was counted manually in each section and mean calculated. 5 samples were viewed from each treatment group and mean number of mast cells and standard deviations were then calculated for each treatment group using Excel software (Microsoft Office).

Transmission Electron Microscopy

Larvae at 8 d.p.f. were fixed in 4% glutaraldehyde in cacodylate buffer containing 0.006% hydrogen peroxide for 3 hours, then washed in cacodylate buffer before post-fixing in osmium tetroxide. Samples were bulk stained with uranyl acetate, dehydrated in ethanol and embedded in Spurr's resin. Thin sections (50 nm) were prepared with a Leica Ultracut UCT, stained with uranyl acetate and lead citrate and viewed in a Philips CM100 electron microscope at 80 KV.

Antibody Labelling

Larvae at 8 d.p.f. were fixed in 4% paraformaldehyde and stained in wholemount as described (Westerfield, 1995) with minor modifications. The TNF-A monoclonal antibody (Abcam) was used at 1:20 dilution; Alexafluor 594 (Molecular Probes) was used as a secondary antibody. Stained larvae were mounted on depression slides and visualised by fluorescence microscopy on a BX51 microscope (Olympus) and images were captured using a ColorView camera and AnalySis software (Olympus). The intensity of TNF-α immunofluorescence was quantified for each treatment group, with a minimum of 5 samples per group, using colour threshold and area measurements in AnalySis (Olympus). Mean values and standard deviations were calculated using Excel software (Microsoft Office).

RESULTS

In vivo observations on gut architecture and motility The optical clarity, speed of development, and fecundity of zebrafish have made them a popular vertebrate model for the study of developmental biology and more recently as an animal model to study disease processes. This optical clarity allows the use of GFP to mark tissues and in the case of bone, a fluorescent dye embryos (Du et al., 2001).

We realized that if we could identify a dye which was swallowed by embryos, but wasn't absorbed and was non-toxic, we would be able to assess gut architecture and function in a live fish.

We have invented an approach to study the disease changes in the gut of zebrafish larvae following the induction of IBD and identified a number of fluorescent compounds that can be used to visualize gut architecture and motility.

Visualisation is achieved by the administration of a fluorescent dye to the embryo medium. The dye is swallowed by the fish but not readily absorbed and therefore fills the entire gut.

When viewed under a fluorescent microscope, the crypts and villi of the gut were apparent as the unstained tissue of the gut wall was highlighted in contrast to the fluorescent medium that filled the gut lumen. In addition, as staining can be performed in vivo, waves of peristalsis were observed (as indicated in the different positions of the gut wall). Of a variety of fluorescent stains used, quercetin was found to be most suitable because of its low toxicity, utility at physiological pH and narrow fluorescence spectrum.

In vivo staining of the gut of live zebrafish larvae was performed at 8 d.p.f. A fluorescent contrast dye was added to the embryo medium and was swallowed by the fish. The contrast dye was not absorbed and hence gave an outline of the gut. Larvae were visualised looking down on the ventral (gut) side. Villi were seen projected into the lumen of the gut. Peristalsis was readily observed. Sites of contraction and relaxation in the gut wall were visible. Larvae were also visualised lying on their sides. In control samples, villi, finger-like projections, were visible protruding into the intestinal lumen. In IBD samples, villus length was reduced and crypts between villi were enlarged. Peristalsis was reduced in IBD samples, therefore the fluorescent contrast dye accumulated in the lumen of the proximal intestine. Transverse sections were haematoxylin and eosin stained. Histological analysis of control samples revealed the presence of crypts and villi in the anterior gut and contraction of the gut lumen. In IBD samples, villi and crypts were absent and the intestinal lumen was enlarged.

Having invented a method for vital staining and visualization of the gut, we coupled this to methods applicable to a variety of diseases, including:

a. assessing gut motility by direct visualization and assessment of peristalsis.;

b. assessing emesis, for example by rapidly creating turbulence through rapid pipetting;

c. inducing damage to the gut to allow assessment of stem cell activity, for example through administration of a chemotherapeutic such as 5FU or radiotherapy;

d. inducing an inflammatory state.

For the latter, we needed a method to induce inflammation throughout the gut in a specific fashion which was easy to administer to allow high throughput screening, and which did not induce generalized inflammation through the entire fish. We discovered that we could achieve these aims through administration of picryl sulfonic acid (PSA, also known as TNBS). It was administered to the embryo medium from 3 d.p.f. to 8 d.p.f. In vivo observations were made daily to identify consequent changes in gut architecture and motility. Abnormalities in villus and crypt morphology and gut motility were first observed at 5 d.p.f. in PSA-exposed larvae and striking disease changes were observed by 8 d.p.f. In control samples, reared in embryo medium, villi were seen to project into the lumen of the gut and quercetin did not accumulate since peristaltic waves moved the contents through the gut. In contrast, following administration of PSA, peristalsis was lost (i.e. an ileus), as in human inflammatory conditions, the gut was dilated and there was reduction in villus length and expansion of crypts.

Histological Appearance of IBD

To ensure that the changes observed in vivo represented disease changes at the cellular level, histological sections were taken through the same area of intestine as examined in vivo. In control samples, the lumen of the gut was small, villi projected into the lumen and narrow clefts were seen between these projections. Following administration of PSA, disease changes observed in vivo were also evident in histological analysis. In fish exposed to PSA, the lumen of the gut was expanded and the lining of the gut was smooth, devoid of villi and clefts. Histological analysis was then performed throughout the length of the gut in control and PSA-exposed fish to determine whether region specific changes could be observed. Reproducible and consistent differences between control and PSA-exposed fish were observed through-out the length of the gut. In the anterior-most intestine, beneath the swimbladder, the epithelium of control samples was characterized by projections and clefts whereas, in PSA-exposed samples the epithelium appeared smooth. This change in histology continued to just posterior to the end of the swimbladder. In the mid-intestine region and extending into the posterior intestine, the gut epithelium appeared normal in control samples with a few goblet cells apparent in the most posterior sections. In PSA-exposed samples, high numbers of goblet cells were observed through-out the mid-intestine and posterior intestine regions. Histological analysis of the rectum and anus did not show any gross changes between control and PSA-exposed samples.

Biological and Pathological Changes in IBD

In rodent studies, IBD induced by TNBS (PSA) is thought to be mediated through a mast cell response (Stein et al., 1998; Xu et al., .2002).

We used a histological stain that specifically labels mast cells (Gurr, 1962), to determine the presence of mast cells in our model. Mast cells were present in the gut of control samples as well as those exposed to PSA, however mast cell infiltration throughout the gut was markedly increased in fish exposed to PSA. We also examined TNFα expression since this protein is important in the initiation and amplification of inflammatory reactions and is a target for modulating excessive inflammatory reactions. Wholemount antibody staining revealed strong staining on the luminal epithelium in PSA-exposed samples whereas staining was absent in controls. To examine the subtle subcellular changes associated with IBD, we examined sections using transmission electron microscopy (TEM). TEM analysis revealed the presence of microvilli on the apical surface of epithelial cells lining the gut in both control and PSA-exposed samples demonstrating that PSA does not cause a chemical burn or damage the epithelial lining of the gut, but causes a more subtle induction of an inflammatory response. In addition, the accumulation of lysosomes towards the lumen in PSA-exposed samples suggests that apical-basal polarity has been maintained. However, the most striking feature of this TEM analysis was the loss of gap and tight junctions between cells in PSA-exposed samples when compared to controls. This is highly reminiscent of the pathology seen in human patients with IBD and suggests that there has been disruption of intestinal barrier function.

Rescue with Prednisolone +5-ASA

Having established that exposure of zebrafish larvae to PSA results in biological and pathological changes relevant to those seen in humans with IBD, we went on to examine the clinical relevance of this model by treating control and PSA-exposed fish with drugs used in human patients with IBD, namely prednisolone and 5-ASA .

A dose/response assay was performed with both prednisolone and 5-ASA in the presence of PSA. In vivo observations of gut architecture were performed at 8 d.p.f. to determine the effective doses of each compound. The effective doses of prednisolone and 5-ASA were then administered to PSA-exposed larvae and histological analysis was performed to determine whether such treatment was able to rescue the disease changes observed with PSA alone.

Both prednisolone and 5-ASA were able to prevent/rescue disease changes when co-administered with PSA and, importantly, both drugs were also effective when administered after the induction of disease changes. In the posterior intestine, prednisolone treatment suppressed the transdifferentiation of goblet cells and the histology in this region appeared overtly normal. However, a quiescent disease phenotype was observed in prednisolone treated samples reminiscent of changes seen in human IBD patients when the disease is in remission.

Haematoxylin and eosin stained transverse sections were examined as control, IBD and prednisolone-treated IBD samples. Matched sections through the anterior gut just distal to the swimbladder were compared, as were matched sections through the posterior gut just anterior to the anus. In the anterior gut, normal samples showed a contracted lumen with villi and crypts present. In IBD samples, crypts and villi were absent and the lumen was enlarged. Normal morphology was observed in IBD fish treated with prednisolone. In the posterior gut, normal morphology was characterised by the absence of goblet cells and a random distribution of nuclei in the contracted walls of the gut. In IBD samples, the many goblet cells were present and the nuclei of the gut epithelium were forced basally. Normal morphology was restored in IBD fish treated with prednisolone. Similar effects were seen with 5-ASA .

Quantification of Anti-inflammatory effects

Having established that prednisolone treatment was able to rescue the disease changes observed following exposure to PSA, we investigated whether TNFA expression and mast cell number could be used to quantify the anti-inflammatory activity of prednisolone at different doses.

Larvae were treated with PSA and varying doses of prednisolone from 3 to 8 d.p.f. Treatment groups were divided in two and processed for TNFα antibody staining or mast cell staining. Both TNFα immunofluorescence levels and mast cell number decreased with increasing doses of prednisolone, demonstrating that anti-inflammatory effects can be quantified.

Selected Compound Screening

An important aim of this invention was to develop a well-validated zebrafish model of IBD amenable to high-throughput screening. Having performed such validation, we have screened a number of compounds in our IBD model. A variety of iNOS inhibitors were tested and demonstrated rescue of the disease phenotype in vivo and in histological analysis. In addition, thalidomide and parthenolide were screened on the basis of their reported anti-TNFα effects (Smolinski and Pestka, Laffitte and Revuz, 2004). Both compounds showed down-regulation of TNFα but failed to rescue the in vivo disease phenotype and failed to show improvement when the gut histology was analysed demonstrating that individual disease indicators can be modulated but overall disease pathology is unaltered with these therapies.

Induction of gut epithelial damage with sudan black in

ethylene

glycol

Stock Solutions

Saturated solution of sudan black made in 70% ethylene glycol (SB/EG).

Stored at room temperature for maximum of 3 months.

Dosing

10 ul SB/EG in 10 ml embryo medium produces epithelial damage after 24 hours. Larvae exposed at this concentration are viable beyond 10 d.p.f.

5 ul SB/EG in embryo medium produces milder, but readily detectable epithelial damage after 24 hours.

Screening Protocol

1) Zebrafish larvae immersed in embryo medium containing 0.05% SB/EG in embryo medium from 3 d.p.f.

2) Red ‘staining’ in the gut is observed 24 hr after treatment.

Staining relates to cell loss from the gut epithelium and bleeding as a result of disrupted intestinal barrier function.

3) ‘Staining’ is observed by viewing anaesthestised embryos under brightfield dissecting scope.

DISCUSSION

Development of a Zebrafish Model of IBD

In developing a zebrafish model of IBD, we have overcome many of the drawbacks known to exist in rodent models. Firstly, since PSA is added to the embryo medium, this ensures equal exposure of all embryos to the same dose without repeated administration. Moreover, the disease state is induced throughout the entire gut and as a result, region-specific disease changes are observed. In contrast, in rodent studies, PSA is most commonly administered by enema or injected into a specific region of the gut and results in only a localised pathology. We observe biological and pathological changes in our disease model that are highly relevant to those seen in the human disease. For example, we have demonstrated TNFα, a key target for many companies interested in anti-inflammatory drugs, is upregulated in our model and can be detected with antibodies raised against the human antigen. In addition, we have validated our model using current therapies used to treat the human disease and can quantify the anti-inflammatory effect using TNFα levels and mast cells numbers.

High throughput screening of phenotypic rescue

In seeking to develop a zebrafish model of IBD, we were not aiming to replace rodent models, rather to develop a vertebrate system amenable to high-throughput screening that can be used to accelerate the steps in drug discovery. Our approach has been to develop a well-validated and relevant disease model in which we can observe the disease phenotype and its rescue and which can be screened rapidly. The importance of looking at the disease phenotype rather than individual disease modulators is demonstrated in our findings following treatment with parthenolide and thalidomide. Both compounds cause down-regulation of TNFα expression in our model, but neither rescues the disease changes observed in vivo or in histological analysis. Indeed, similar effects have been observed in rodents, where parthenolide can reduce TNFα levels in vitro but fails to have an effect in LPS-induced inflammation in vivo (Smolonski and Pestka, 2003). A further benefit of our model is the ability to quantify anti-inflammatory effects, as demonstrated with different doses of prednisolone. Such quantification will allow compounds to be ranked according to potency.

The invention also allows for the assessment of nausea, vomiting and gut motility.

### EXAMPLE 1

A 7 dpf embryo is given food granules of a size suitable for a 14 dpf fish. Typically the embryo attempts to ingest this food, but then regurgitates it out. By measuring the amount of food which is regurgitated and the rate it is possible to assess the emetic state of the fish. This may be further facilitated by labeling the food, for example with a fluorescent, colourimetric or radioactive label.

### EXAMPLE 2

A 7 dpf embryo is given food granules of an appropriate size. Typically the embryo attempts to ingest this food with little difficulty. If the embryo was prone to vomiting, or was in the presence of an agent which was being assessed for its potential emetic properties, by measuring the amount of food which is regurgitated and the rate it is possible to assess the emetic state of the fish. This again may be further facilitated by labeling the food, for example with a fluorescent, colourimetric or radioactive label.

### EXAMPLE 3

The gut of a 7 dpf embryo is labeled through use of a dye, as described earlier. Nausea is then induced in the embryo through administration of an emetic such as ipecacuahna or a chemotherapeutic, or by using a pipette, such as a 2.5 ml plastic pipette commonly used for transferring embryos, to rapidly suck up then expel the fish water to create currents and therefore movements of the embryos to induce vomiting. This then leads to vomiting of the dye in the gut. This may be directly measured through visualization under a fluorescent dissecting microscope. Regurgitated label can be visualized in the surrounding medium, or absence of the label in the intestine can be used as a measure of the amount of label vomited. Alternatively a fluorescent, colourimetric or radioactive label may be utilized to facilitate quantitation and high throughput of this assessment.

This may then be used in scenarios such as:
a. Administer a compound to assess whether this has any emetic effect in the normal state.
b. Administer a compound to assess whether this has any antiemetic effect.

### EXAMPLE 4

The rate at which food is moved from the gut into the small intestine is an important consideration for many drugs. By labeling the food, or the gut it is possible to directly assess the time it takes food to pass from the stomach into the intestine. Ideally this is carried out with the use of a video camera to enable quantitation of the number of peristaltic waves, their coordination and the size of the intestinal lumen. Comparing the effect of a test compound on the normal gut motility assessed by these methods, or during a state of nausea (e.g. induced as described above), then enables the identification of pro-kinetic agents.

The model is also suitable for the assessment of inflammation in general. As well as looking at the in vivo gut function, as a measure of the disease state in a live animal, it is also possible to assess the extent of inflammation by other methods, including the following.

The animals may be fixed, sectioned and stained with a histological stain such as Haematoxylin and Eosin. It is then possible to quantify the degree of inflammation. For example, the following graded changes were seen with one test drug administered following induction of the inflammatory state:

1 ug/ml—no rescue observed from histological analysis.

2.5 ug/ml slight improvement in villus and crypt morphology, but no overt improvement in number or region of transdifferentiation of goblet cells.

0.5 ug/ml—normal crypt and villi morphology, but goblet cell phenotype not fully rescued (some improvement observed).

10 ug/ml—histology appears completely normal but quiescent phenotype noted by external validator.

Further assessment may involve immunhistochemical analysis. For example we have detected upregulation of TNFalpha in the disease state, with a graded decrease in the extent of this upregulation during co-administration of an anti-inflammatory agent. This was quantified by measuring the relative fluorescence of the signal emitted by the 2ry antibody binding to the primary antibody. The latter was raised against human TNFαalpha. We have shown that this cross-reacts with zebrafish TNFalpha. Antibodies raised against other inflammatory mediators may also be used.

Alternatively, a histochemical stain versus a particular inflammatory cell type may be used. For example, mast cells may be stained, as described herein. It is then easy to see the mast cells lining the gut in a sagittal section. The total number of mast cells in the gut may then be counted in the entire fish to enable a quantified of the degree of inflammation. Similarly, a histochemical stain that marks non-inflammatory cells that we have demonstrated are upregulated in the inflammatory state e.g. alcian blue for the detection of mucin-rich goblet cells.

Mucosecretory diseases involve proliferation of goblet cells and excessive secretion of mucin. These include conditions of the respiratory system, such as obstructive airways disease. The gut biology assessment and inflammatory bowel disease model, disclosed herein, have direct applicability to mucosecretory diseases in general. Specifically, the presence and activity of goblet cells provides a means of addressing one aspect of mucosecretory diseases. For example, in the inflammatory state disclosed herein, goblet cells are seen to extend across a larger region of the gut than normal and are larger in size, and are more numerous in regions of the gut where goblet cells are normally present. Thus by measuring the number and size of the goblet cells, for example in the presence of a test agent, it is possible to assess the effect of that test agent of the biology of the goblet cells. These goblet cells may be visualized and quantified by means of standard histological stains, such as the H&E stain as described herein. Alternatively, antibodies binding to components of the goblet cells may be used to facilitate a quantification of the extent of the goblet cells. A further method involves a stain for the production of mucin, or the creation of a transgenic line in which a fluorescent marker such as GFP is expressed in mucin producing cells such as goblet cells through the use of a tissue specific promoter.

Metaplasia involves the conversion of a tissue from one cell type to another. For example in gut metaplasia, the normal columnar epithelial cell lining may be replaced by a squamous cell epithelium. Such metaplastic changes are important clinically as they are precancerous. The inventors have realized that their gut biology assessment and in particular their inflammatory bowel disease model, disclosed herein, has direct applicability to metaplasia, and cancerous diseases in general. Specifically, the alteration of the lining of parts of the gut which do not normally contain goblet cells to a lining containing goblet cells provides a means of addressing metaplastic disease. The assessment of the extent of this change has been carried out successfully by the inventors as described elsewhere herein.

Abnormalities in gut motility are a major cause of morbitiy. In particular, a significant underlying component of irritable bowel syndrome is disordered gut motility. The inventors have realized that components of their invention are directly applicable to the assessment of gut motility. For example, the gut of a normal 7 dpf embryo is labeled as described herein. The peristaltic waves are then visualized in the live fish by means of a fluorescent microscope. A digital CCD camera may be used to record and quantify the normal motions of the bowel. In particular the coordinated contractions of the bowel, with the progression of the wave of bowel contraction from proximal to distal bowel is assessed, as well as the frequency, amplitude and velocity of this movement. The fish is then exposed to compounds known to alter gut motility, such as mebeverine and evening primrose oil to compare the pattern of contraction with the normal embryo. Alternatively a novel test compound, designed to improve or alter an aspect of gut motility, may be administered to the fish and assessed.

The output of faeces or labeled compound from the gut may be measured, for example through colourimetric, radiolabelling, or weighing.

The transit time from ingestion of a compound to emergence in the faeces may be measured.

These methods are also applicable to the identification of an anti-diarrhoeal or a purgative. For example, the fish may be exposed to loperamide, to decrease the transit time, followed by coadministration of the test purgative.

These methods are also applicable to the assessment of satiety, appetite and absorption. For example, the amount of faeces produced by the fish may be measured tp provide a quantitative assessment of the amount of food ingested, or the amount of food absorbed. By offering the fish different foods, labeled in different ways, selectivity of food intake may also be measured.

The inventors have additionally realized that their model provides an excellent assessment of gut stem cell activity, and indeed in vivo stem cell activity in general. Embryos were exposed to BrdU (bromo-deoxyuridine), administered directly to the fish water. Fluorescent cells, indicative of a dividing cell during exposure to the BrdU, were seen lining the gut. By measuring the number of such cells it is possible to get a measure of stem cell activity in the normal fish in a quantitative fashion. The activity of these stem cells may then be affected through the administration of a chemotherapeutic, such as 5-fluorouracil, vincristine or vinblastine, or through radiotherapy. The susceptibility of the stem cells to these agents, as well as the rate of recovery of the cells and repopulation of the damaged gut following removal of these chemotherapeutic agents, may also be assessed. This provides a means for identifying stem cell activators, as well as drugs with potentially harmful bowel side effects. The inventors have already demonstrated, through administration of sudan black and ethylene glycol, that it is possible to induce a chemotoxic injury to the gut.

As well as assessing the stem cell activity and extent of repopulation of the gut wall through BrdU labeling, it is also possible to assess this through the hisological and immunhistochemical methods described elsewhere herein, or adaptations thereof (e.g. peanut lectin).

### REFERENCES

Barut and Zon (2000). Physiological Genomics (13): 49-51.

Calkins and Mendelodd (1995). Inflammatory Bowel Disease (4th Edition). Kirsner, J. B. and Shorter, R. G. (eds). Williams and Wilkins, Baltimore.

Colas et al. (1996). Nature (380): 548-550.

Dooley and Zon (2000). Current Opinions in Genetics and Development (10): 252-256.

Du et al. (2001). Developmental Biology (238): 239-246.

Fishman (2001). Science (294): 1290-1291.

Golling et al. (2002). Nature Genetics (31): 135-140.

Gurr (1962) Staining Animal Tissues: Practical and Theoretical. Leonard Hill Books, London.

Kimmel et al. (1995). Developmental Dynamics (203): 253-310.

Mueller C. (2002). Immunology (105): 1-8.

Parkes and Jewell (2001). Expert Reviews in Molecular Medicine (19 November), http://www-ermm.cbcu.cam.ac.uk/0100391Xh.htm

Raz et al. (1998). Current Biology (8): 82-88.

Remington's Pharmaceutical Sciences (1980). 16th Edition, Osol, A. (ed). Mack Publishing Company, USA.

Smolinska and Pestka (2003). Food and Chemical Toxicology (41): 1381-1390.

Solnica-Krezel et al. (1994). Genetics (136): 1401-1420.

Stein et al. (1998). American Journal of Physiology (274): G203-G209.

Watts and Satsangi (2002). Gut (50):iii31-iii36.

Westerfield (1995). The Zebrafish Book. University of Oregon Press. Eugene, OR

Xu et al. (2002). Scandinavian Journal of Gastroenterology (3): 330-337.

1. A fish model for gut inflammatory disease, wherein a pro-inflammatory agent trinitrobenzene sulfonic acid (TNBS) or dextran sulphate sodium (DSS) is administered to medium containing embryonic fish to induce the disease state in the fish gut.

2. A fish model according to claim 1 wherein the pro-inflammatory agent is trinitrobenzene sulfonic acid (TNBS).

3. A fish model according to claim 1, wherein the fish is optically clear and a non-toxic, non-absorbed fluorescent dye or contrast medium is administered to the medium to allow visualization of the fish gut following swallowing of the dye by the fish.

4. A fish model according to claim 1, wherein the fish has inflammatory bowel disease.

5. A fish model for screening for gut function and/or disease phenotype, wherein a non-toxic, non-absorbed fluorescent dye is administered to medium containing embryonic fish that are optically clear to allow visualization of the fish gut following swallowing of the dye by the fish.

6. A fish model according to claim 1, wherein the fish is a zebrafish.

7. A method of screening for a compound for use in treatment of gut inflammatory disease, comprising:
treating a fish model according to claim 1, with a compound, identifying a compound that treats the gut inflammatory disease in the fish model.

8. A method according to claim 7, comprising high-throughput screening.

9. A method according to claim 7 comprising screening for optimal combinations of possible anti-inflammatory agents.

10. A method of screening for a genetic mutation that rescues or suppresses disease phenotype of a gut inflammatory disease, comprising:
mutating a fish model according to claim 1,
identifying a mutation in the fish model that rescues or suppresses the gut inflammatory disease phenotype.

11. A method according to claim 7, comprising histological analysis of the gut of the fish model to determine effect of a compound or mutation on the gut inflammatory disease in the fish model.

12. A method of generating a fish model for gut inflammatory disease, the method comprising administering a pro-inflammatory agent trinitrobenzene sulfonic acid (TNBS) or dextran sulphate sodium (DSS) to medium containing embryonic fish to induce the disease state in the fish gut.

13. A method according to claim 12 wherein the pro-inflammatory agent is trinitrobenzene sulfonic acid (TNBS).

14. A method according to claim 12, wherein the fish is optically clear and a non-toxic, non-absorbed fluorescent dye or contrast medium is administered to the medium to allow visualization of the fish gut following swallowing of the dye by the fish.

15. A method of generating a fish model for screening for gut function and/or disease phenotype, the method comprising administering a non-toxic, non-absorbed fluorescent dye to medium containing embryonic fish that are optically clear to allow visualization of the fish gut following swallowing of the dye by the fish.

16. A method according to claim 12, wherein the fish is a zebrafish.

17. A method for assessing nausea, vomiting and/or an anti-emetic, an emesis inducing compound, the method comprising:
administering to a fish that is optically clear a non-toxic, non-absorbed fluorescent or otherwise labeled compound which is ingested,
administering to the fish the emesis inducing compound and/or inducing nausea through pipetting or rapid movement,
assessing movement of the labeled compound through the gut of the fish, or its re-emergence from the mouth of the fish.

18. A method for assessing inflammation, the method comprising:
using a fish model of claim 1, or inducing an inflammatory state in the gut of a fish by administration of a pro-inflammatory agent TNBS or DDS to the medium containing the fish or by genetic manipulation; and
assessing the state of the gut by:
conducting a whole mount or sectioned histological analysis, together with a general histological stain such as Haematoxylin and Eosin, or a specific stain for, for example, mast cells, such as ethanol-based toluidine blue for the detection of mast cells, or alcian blue for the detection of mucin and mucin-secreting cells (e.g. goblet cells).
carrying out immunohistochemical analysis for a marker of the state of the gut such as an antibody stain for TNFalpha, or a stain for one or more interleukins and/or cytokines known to be up-regulated in inflammatory conditions,
carrying out biochemical assessment of the gut, for example through assay of TNFalpha levels, and/or carrying out histological, immunological or gene expression analysis for upregulated proliferation or increased cell death as a response to the induction of inflammation and the suppression of an inflammatory response.

19. A method for assessing mucosecretory disease, the method comprising:
using a fish model of claim 1, or inducing an inflammatory state in the gut of a fish by administration of a pro-inflammatory agent TNBS or DDS to the medium containing the fish or by genetic manipulation; and
analyzing normal gut and/or inflammatory gut in a fish, with
assessment of the extent, number and/or activity of goblet cells, for example by a method comprising:
conducting a whole mount or sectioned histological analysis, together with a general histological stain such as Haematoxylin and Rosin, or a specific stain for, for example, goblet cells or mucin, e.g. alcian blue PAS staining,
generating a cell or tissue specific transgenic line expressing a marker, such as GFP, for, for example, goblet cells or mucin, e.g. MUC2, a differentiation marker of the goblet cell lineage, or cytokeratin-7, a goblet-cell specific cytokeratin,
conducting a whole mount or sectioned immunohistological analysis, using an antibody to specifically label goblet cells e.g. MUC2 or cytokeratin-7, and/or
conducting analysis of cell proliferation studies (e.g. BrdU labeling or immunohistochemistry for proliferating cell nuclear antigen, PCNA).

20. A method for assessing a cancerous state, metaplasia and/or transdifferentiation, the method comprising:
using a fish model of claim 1, or inducing an inflammatory state in the gut of a fish by administration of a pro-inflammatory agent TNBS or DDS to the medium containing the fish or by genetic manipulation; and
analyzing in normal gut and/or inflammatory gut in a fish, with assessment of the extent number and/or activity of cell types in the gut, by a method comprising:
conducting a whole mount or sectioned histological analysis, together with a general histological stain such as Haematoxylin and Eosin, or a specific stain for, for example, goblet cells or mucin, e.g. alcian blue PAS staining, and/or
generation of a cell or tissue specific transgenic line expressing a marker, such as GFP, for, for example, goblet cells or mucin, e.g. MUC2 or cytokeratin-7.

21. A method for assessing gut motility, IBS, diarrhea or constipation, the method comprising:
administering to a fish a fluorescent- or otherwise-labeled compound which is ingested or directly visualizing the gut,
optionally administering to the fish a motility modifying compound,
assessing movement of the labeled compound through the gut, or movements of the gut itself, or emergence of the labeled compounds or of feces from the anus, and/or assessing quantity and quality of the feces.

22. A method for assessing stem cell activity, the method comprising:
visualizing cell turnover and differentiation in a fish gut through:
direct visualization of gut architecture through in vivo assessment or histological analysis, and/or BRDU labeling, or other labeling of mitotic activity,
in the normal state, or following administration of a chemotoxin, cytotoxin or radiotherapy.

23. A method for assessing the effects of cancer therapies, the method comprising:
visualizing cell turnover and differentiation in a fish gut through:
direct visualization of gut architecture through in viva assessment or histological analysis, and/or BRDU labeling, or other labeling of mitotic activity
in the normal state, or following administration of a chemotoxin, cytotoxin or radiotherapy.

24. A method according to claim 15 comprising treating the fish with a test compound and observing an effect.

25. A method according to claim 15 comprising mutating the fish model and observing an effect.

26. A fish model according to claim 1 wherein the pro-inflammatory agent is dextran sulphate sodium (DSS).

27. A method according to claim 12 wherein the pro-inflammatory agent is dextran sulphate sodium (DSS).

28. A fish model for gut damage, wherein a chemotoxic agent sudan black in ethylene glycol or a cytotoxic agent is administered to medium containing embryonic fish to induce the disease state in the fish gut.

29. A method of generating a fish model for gut damage, the method comprising administering a chemotoxic agent sudan black in ethylene glycol or a cytotoxic agent to medium containing embryonic fish to induce damage in the fish gut.
